(19) United States
(12) Patent Application Publication
(10) Pub. No.: US 2004/0220636 A1
(43) Pub. Date: Nov. 4, 2004

(54) Cardiac pacing therapy parameter programming

(75) Inventor: John E. Burnes, Andover, MN (US)

Correspondence Address: MEDTRONIC, INC., 710 MEDTRONIC PARKWAY NE, MS-LC340, MINNEAPOLIS, MN, 55432-5604, US

(73) Assignee: Medtronic, Inc.

(21) Appl. No.: 10/426,645

(22) Filed: Apr. 29, 2003

(51) Int. Cl. A61N 1/365

(52) U.S. Cl. 607/17

(57) ABSTRACT

Accordingly, according to the present invention a programmable IMD provides a patient with an essentially customized cardiac pacing therapy resulting in enhanced hemodynamic function. In particular, the present invention provides for refined tuning of pacing parameters to cause the heart to pump blood and perfuse in an efficient manner. In general, the invention promotes good hemodynamic operation through programming of an implantable medical device (IMD) as a function of one or more hemodynamic data sensed by a device located external to the body of the patient relying on said data as gathered either by discrete internal measuring device or an external device. The ability to share data among and between an IMD, an IMD programming device and a hemodynamic monitoring or measuring device spaced from the IMD (i.e., either an implantable device or external to the patient) allows for improved selection of pacing parameters to optimize hemodynamic function.

### CROSS REFERENCE TO RELATED APPLICATIONS

This patent disclosure hereby incorporates by reference the following patent applications filed on even date hereof; namely, P-11214, “Method and Apparatus for Detecting Myocardial Electrical Recovery and Controlling Extra-Systolic Stimulation; P-11216, “Method and Apparatus to Monitor Pulmonary Edema; P-11252, “Method and Apparatus for Determining Myocardial Electrical Resitution and Controlling Extra Systolic Stimulation; and P-11215, “Use of Activation and Recovery Times and Dispersions to Monitor Heart Failure Status and Arrhythmia Risk”.

### TECHNICAL FIELD

The invention relates to cardiac pacing systems, and more particularly to programmable cardiac pacing systems and automatic optimization of pacing parameters by iteratively altering one or more such pacing parameters and capturing at least one hemodynamic response resulting therefrom with an external and/or an internal sensing or measuring device.

### BACKGROUND

Many patients receive an implantable medical device (IMD), such as a pacemaker, an implantable cardioverter-defibrillator, and the like that addresses abnormal cardiac rates or rhythms. One common type of IMD is a pacemaker that senses cardiac activity such as single or multiple chamber depolarization (i.e., left or right atrial and/or ventricular activity) and delivers timed electrical stimulation therapy to activate one or more atria or ventricles. Typically, one or more deployable medical electrical leads (or other electrodes) coupled to such an IMD senses an atrial activation or causes an atrial activation with an electrical pacing stimulus and after a predetermined time interval provides pacing stimulus to one or both ventricles.

Some patients, such as those suffering from heart failure, develop a wide QRS complex resulting from a delayed activation of one of the ventricles in the heart, and inter- and/or intra-ventricular electrical-mechanical dysynchrony. Such dysynchrony may worsen heart failure symptoms. For example, the patient may experience a reduction in cardiac output because the ventricles begin or complete contracting at significantly different times. The timing imbalance may also cause the patient to experience paraoxysmal septal motion, mitral regurgitation and/or inadequate atrial contribution to ventricular filling, and the like.

Patients having a wide QRS complex or having inter- and/or intra-ventricular electrical-mechanical dysynchrony appear to benefit from therapy provided by synchronized pacing therapy provided to both ventricles. This particular tyupe of pacing therapy has become known as cardiac resynchronization therapy (CRT). In one generic form of CRT electrodes operably coupled to IMD circuitry sense (or pace) atrial chamber activity, and then after a predetermined time interval after each sensed or paced atrial activation, provide synchronized bi-ventricular pacing therapy. Accordingly, each ventricular chamber may be paced simultaneously, or one ventricle may be paced before another. When one ventricle is paced before the other, the time delay between ventricular paces is generally known as a V-V interval. This bi-ventricular pacing is one form of cardiac resynchronization, and it presently improves the quality of life, exercise capacity and overall cardiac function for many heart failure patients. Cardiac resynchronization therapy may also be applied to the atria. The atria may be paced simultaneously, or one atrium may be paced before the other. When one atrium is paced before the other, the time delay between atrial paces is generally known as an A-A interval. These intervals, among others, represent pacing parameters and adjustment of one of more of such intervals can have disparate effects on hemodynamic function.

Due in part to the importance of improving hemodynamic function, particularly for heart failure patients, the present invention provides a method and apparatus for applying select pacing parameters and pacing parameter combination to enhance hemodynamic function.

### SUMMARY

Accordingly, according to the present invention a programmable IMD provides a patient with an essentially customized cardiac pacing therapy resulting in enhanced hemodynamic function. In particular, the present invention provides for refined tuning of pacing parameters to cause the heart to pump blood and perfuse in an efficient manner. In general, the invention promotes good hemodynamic operation through programming of an implantable medical device (IMD) as a function of one or more hemodynamic data sensed by a device located external to the body of the patient relying on said data as gathered either by discrete internal measuring device or an external device. The ability to share data among and between an IMD, an IMD programming device and a hemodynamic monitoring or measurement device spaced from the IMD (i.e., either an device implanted within or external to the body of a patient) allows for improved selection of pacing parameters to optimize hemodynamic function.

In a typical application, a programmer sets a pacing parameter in the IMD. An external device monitors the patient while the IMD applies the pacing parameter, and generates hemodynamic data indicative of hemodynamic function. By example and without limitation, representative hemodynamic data include (or can be derived from) stroke volume, cardiac output, heart rate, ECG/EGM, heart sounds, blood pressure, blood flow, temperature, tissue impedance, trans-thoracic impedance, body fluid analysis (e.g., saturated oxygen, carbon dioxide, pH, lactate), tissue saturation (e.g., relating to pulmonary edema and the like), circulation delay time (e.g., a time period from an initial stimulation or perturbation of the cardiovascular system to detection of a corresponding response), cardiac tissue contractility index, mechanical restitution (MR), recirculation fraction (RF), ejection fraction, acceleration or movement of various parts of the heart (e.g., portions of atrial or ventricular wall tissue, septal wall tissue, and the like), volumetric (or dimension) data for a heart during the cardiac cycle, and various dedicated left- and right-side hemodynamic measurements as is known in the art, but as used herein, hemodynamic data encompasses any metric that reflects or relates to actual hemodynamic function. In addition, first and second derivatives and integrals of the foregoing may be used to derive or integrate primary measurements, respectively, to produce additional hemodynamic data useful when practicing the present invention.

Furthermore, in the context of the present patent disclosure, the rubric of “hemodynamic data” includes without limitation data reflecting cardiac mechanical function. For example, contractility metrics as measured by diverse sensors such as accelerometer(s) adapted to be coupled to endo-, epi- or peri-cardial tissue, blood pressure sensors, fluid flow sensors, and the like may be used to adjust pacing parameters according to the present invention. Dispersion of depolarization wave fronts (and corresponding wave backs) through and around features of a patient's cardiac physiology as measured by pacing, defibrillation electrodes and/or other electrodes disposed around or near the heart.

Accordingly, at least one piece of said hemodynamic data is utilized when practicing the present invention; however, in one embodiment of the invention a plurality of such data is used to select pacing parameters to optimize hemodynamic function. Also, collection of such data may occur over a very short period of time and/or may represent longer-term trend information although such data collection typically lags any changes to one or more pacing parameters by at least a few minutes so that any transient effects are minimized.

From time to time in the present patent disclosure intrinsic cardiac events are distinguished from evoked cardiac events. Thus, the phrase “pacing parameter” is meant to comprehend myriad pacing parameters, including timed, device-related cardiac pacing intervals (e.g., A-A, V-V, A-V, V-A, etc.) and intrinsic intervals (e.g., P-P, P-R, R-R, R-P, etc.) and combinations thereof (e.g., A-R, P-V, R-A, V-A, etc.) and the like. The phrase “pacing parameters” is also intended to include intrinsic heart rate information (typically expressed as beats-per-minute or bpm) as well as paced heart rate (expressed as paces-per-minute or ppm) and intervals related thereto. For example, programmed sensing intervals (e.g., SAV or “sensed A-V” interval, PAV or “paced A-V” interval, and the like), and blanking periods (e.g., PVAB or “post-ventricular atrial blanking”) and programmable refractory periods (e.g., PVARP or “post-ventricular atrial refractory period”) and the like. Further, in this disclosure pacing stimulus information such as stimulus amplitude, duration, and waveform type (e.g., mono-, bi- or multi-phasic, etc.) and/or rate and the like are also included under the rubric of “pacing parameters.” In addition, the phrase is intended to include the so-called pacing modality or schema (e.g., DDD, VVI, ADI, AAI, VOO, etc.) as well as rate-responsive derivatives thereof. As mentioned previously, the phrase is also intended to cover pacing modalities such as CRT (or bi-ventricular therapy) as well as the relatively new pacing modality becoming known as minimum ventricular pacing (MVP) therapy. One form of MVP therapy involves periodically confirming intact AV conduction and delivering atrial-biased pacing therapy (e.g., AAI/R or ADI/R) and changing to dual chamber or ventricular pacing therapy if AV conduction is lacking (e.g., DDD/R, DDI/R, VVI, etc.). Moreover, the phrase pacing parameters includes newly re-emerging pacing modalities and related parameters such as those related to paired or coupled pacing therapy (also known as post extra-systolic potentiation therapy or PESP therapy) which include the notion of an extra-systolic interval (ESI) for the time period between a ventricular pace or an intrinsic ventricular depolarization and an electrical augmentation stimulus delivered shortly after the relative refractory period of said ventricle. The PESP therapy may include additional parameters a cardiac stress index (CSI), a cardiac performance index (CPI) and diverse other pacing parameters. With respect to PESP, the following references are incorporated by reference herein, U.S. Pat. No. 6,213,098 to Bennett et al. and assigned to Medtronic, Inc. and non-provisional U.S. patent application Ser. No. 10/232,792 (Atty. Dkt. P-9854.00) filed 28 Aug. 2002. The phrase pacing parameters can without limitation also include anti-arrhythmia therapy parameters such as anti-tachycardia pacing (ATP), electrical stimulation metrics for atrial or ventricular cardioversion and/or defibrillation (e.g., a pacing threshold, a defibrillation threshold, a cardioversion threshold). Finally, the phrase pacing parameters without limitation includes timing of intrinsic arrhythmic events such as one or more premature atrial or ventricular contraction (PAC or PVC, respectively), ventricular tachycardia (VT), ventricular fibrillation (VF), atrial fibrillation (AF), and the like.

In practicing the present invention, an IMD programming device receives at least one piece of hemodynamic data from one or more hemodynamic measurement device (one or more external and/or co-implantable devices), and programs one or more pacing parameters of the IMD as a function of one or more pieces of the hemodynamic data. The IMD programming device, of course, is also telemetrically linked to the IMD and may read, write or store virtually any pacing parameter of the IMD to the IMD and/or to the IMD programming device. The hemodynamic measurement device continues to monitor the patient and generates an updated piece of hemodynamic data, and the programmer may set the one or more pacing parameters again as a function of the updated hemodynamic date.

In addition, the IMD may communicate physiologic data and/or pacing parameter settings to the hemodynamic measuring device(s). Thus, the duty cycle or timing for any measurements can be efficiently enhanced for example, by triggering data collection to a predetermined time interval when the measurement is most readily or efficiently taken. In the event that one or more of the measuring device(s) are also implanted such efficiency results in conservation of the power source for such device(s) while limiting the amount of filtering required to produce usable hemodynamic data, among other advantages.

In this way, an IMD programming device may iteratively program one or more pacing parameters of an IMD. When the hemodynamic measurement device generates a hemodynamic datum indicating that the hemodynamic operation of the patient appears optimized (or at least satisfactory), the IMD programming device establishes said one or more pacing parameters by programming the IMD accordingly.

In one embodiment, the invention is directed to a method comprising receiving at least one hemodynamic data from a hemodynamic measuring device (e.g., a device external to or implanted within the body of a patient) and setting a pacing parameter in an IMD as a function of the hemodynamic data. In another embodiment, the invention is directed to a method comprising setting a pacing parameter in an implantable medical device to a first setting and receiving hemodynamic data from a hemodynamic measuring device while the pacing parameter is at the first setting. In further embodiments, the invention is directed to a computer-readable medium containing instructions that cause a programmable processor to carry out any of the foregoing methods.

In an additional embodiment, the invention is directed to processor-based devices. The processor receives a hemodynamic data item from a device implanted in the body of a patient or from an external device, or both, and generates a pacing parameter setting for the implanted device as a function of the hemodynamic data.

In another embodiment, the invention provides a hemodynamic measurement device that is wholly external to the body of a patient. The device comprising a sensor to measure a hemodynamic datum from a body of a patient and a transmitter to transmit the hemodynamic datum to an IMD programmer. As mentioned previously, the operation of the hemodynamic measurement device is optionally enhanced by receiving information, including pacing parameter information, from an IMD so that the hemodynamic measurement device more efficiently and/or accurately measures hemodynamic function of the patient.

In an added embodiment, the invention is directed to a system that includes a sensing device, a programmer, and an implantable medical device. The sensing device is external to the body of the patient and measures a hemodynamic datum. The programmer, which may be external or implanted, generates a pacing parameter setting as a function of the hemodynamic datum. The implantable medical device applies pacing stimuli to a heart according to the pacing parameter setting.

The details of one or more embodiments of the invention are set forth in the accompanying drawings and the description below. Other features, objects, and advantages of the invention will be apparent to those of skill in the art to which the present invention is directed upon review of the written description, drawings, and claims appended hereto.

### BRIEF DESCRIPTION OF DRAWINGS

FIG. 1 is a block diagram illustrating an exemplary system that may practice the invention, including an external device, a programmer and an IMD.

FIG. 2 illustrates an exemplary IMD that may be programmed using the techniques of the invention, located in and near a heart.

FIG. 3 is a functional schematic diagram of the embodiment of the IMD shown in FIG. 2.

FIG. 4 is a schematic diagram illustrating an external device for measuring an exemplary hemodynamic datum, transthoracic impedance.

FIG. 5A is a timing diagram illustrating an exemplary relation between an exemplary hemodynamic datum, cardiac output, and an exemplary pacing parameter, a V-V interval.

FIG. 5B is a timing diagram illustrating a change in the exemplary hemodynamic datum resulting from a change to the exemplary pacing parameter shown in FIG. 5A.

FIG. 6 is a flow diagram illustrating an exemplary technique for monitoring a hemodynamic datum and programming an IMD according to an embodiment of the invention.

### DETAILED DESCRIPTION

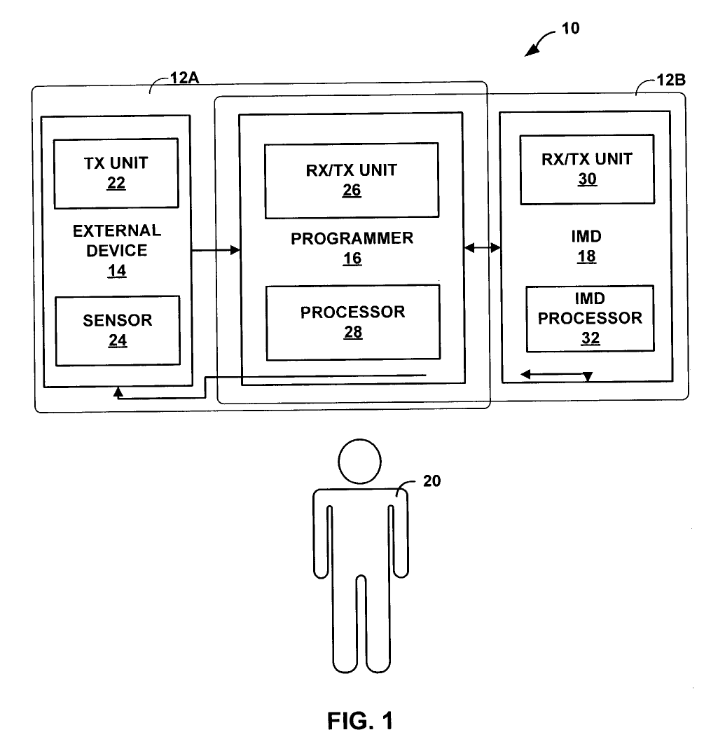

FIG. 1 is a block diagram illustrating an example embodiment of a system 10 including an external device 14, a programmer 16 and an implantable medical device (IMD) 18. System 10 is shown in FIG. 1 with a patient 20. In one form of the present invention the external device 14 is external to the body of patient 20, and in another form of the invention the device 14 may comprise a fully- or partially-implantable device. In both forms of the present invention, the device 14 includes a sensor 24 that senses or measures hemodynamic function of the heart of patient 20. More particularly, the device 14 generates at least one hemodynamic datum relating to the hemodynamic function, and transmits a signal reflecting the hemodynamic datum via a transmitter unit (TX unit) 22 to programmer 16. The TX unit 22 may comprise circuitry to establish a wireless or hard-wired communication link with programmer 16. In addition, mutual communication among the device 14 (whether disposed outside, partially outside or implanted within the patient), the programmer 16 and the IMD 18 is preferably established intermittently or continuously. According to the present invention, such mutual communication enhances the process of optimizing the programming of the IMD 18 and the resulting hemodynamic function of the patient. Furthermore, such mutual communication enhances the operation of device 14, as more fully described herein.

The phrase “hemodynamic data” covers any information that relates to one or more discrete characteristics or metrics of the hemodynamic and/or mechanical function of the heart. Typical hemodynamic data include stroke volume, cardiac output and heart rate, as well as any of the factors enumerated above in the Summary of the invention that vary as a reflect directly or indirect on hemodynamic and/or mechanical function of a patient. The present invention is intended to encompass all factors that vary as a function of hemodynamic operation and/or mechanical function of a patient.

The device 14 comprises any external and/or co-implanted sensor that generates a signal in response to hemodynamic operation or mechanical cardiac function. The device 14 may comprise, for example, an impedance monitor that that measures transthoracic impedance. As will be described in more detail below, transthoracic impedance varies as a function of cardiac output. Device 14 may generate a hemodynamic datum based on one or more transthoracic impedance measurements, thereby providing a measurement of stroke volume.

Of course, the device 14 is not limited to a transthoracic impedance monitor, and may comprise one or more other sensors that generate one or more signals in response to hemodynamic operation. Furthermore, for convenience the device 14 will primarily be referred to as an “external device” although as previously mentioned the present invention is not to be construed as limited only to non-implanted devices. The device 14 may comprise, for example, a heart rate sensor, a heart sounds sensor, a blood pressure sensor, a blood flow sensor, and other apparatus as more fully set forth in the Summary, and the like. Heart rate, heart sounds, blood pressure and blood flow directly and/or indirectly reflect hemodynamic operation or mechanical function. Furthermore, the device 14 is not limited to a single sensor, but may include any combination of sensors that generate signals in response to hemodynamic operation. The device 14 preferably includes circuitry to telemeter data to and from the programmer 16 and the IMD 18, including timing circuitry so that the device 14 can make relatively synchronized hemodynamic and other measurements. Also, for some types of telemetry it is possible that interference arising from a device 14 (e.g., an impedance monitor) thereby corrupting telemetry operation. In the event that such interference occurs, appropriate timing or multiplexing techniques and the like may be used to reduce or eliminate such interference.

Programmer 16 receives the hemodynamic datum from external device 14 via a receiver/transmitter unit (RX/TX unit) 26. A processor 28 in programmer 16 sets one or more pacing parameters as a function of one or more hemodynamic or mechanical data. In general, the phrase pacing parameter is meant to cover all parameters that govern delivery of electrical stimulation therapy to one or more chambers of the heart of a patient 20. Pacing parameters may govern, for example, the rate or timing of pacing stimuli. Exemplary pacing parameters include one or more pacing intervals, such as an A-V interval, a V-V interval, and an A-A interval.

Programmer 16 programs IMD 18 with the pacing parameter. In particular, programmer 16 transmits the pacing parameter setting to IMD 18 via RX/TX unit 26, and IMD 18 receives the pacing parameter setting via a receiver unit (RX unit) 30. Typically, RX/TX unit 26 in programmer 16 comprises circuitry to establish a wireless communication link with RX unit 30.

IMD 18 further includes a processor 32 that implements the pacing parameter setting. In other words, the pacing parameter setting is an instruction from programmer 16 to IMD 18 that directs the pacing of IMD 18. IMD 18 implements the pacing parameter setting by applying pacing stimuli to the heart of patient 20, according to the pacing parameter setting. When the pacing parameter setting specifies a time duration for an A-V interval, for example, IMD 18 paces the heart of patient 20 with the specified A-V interval.

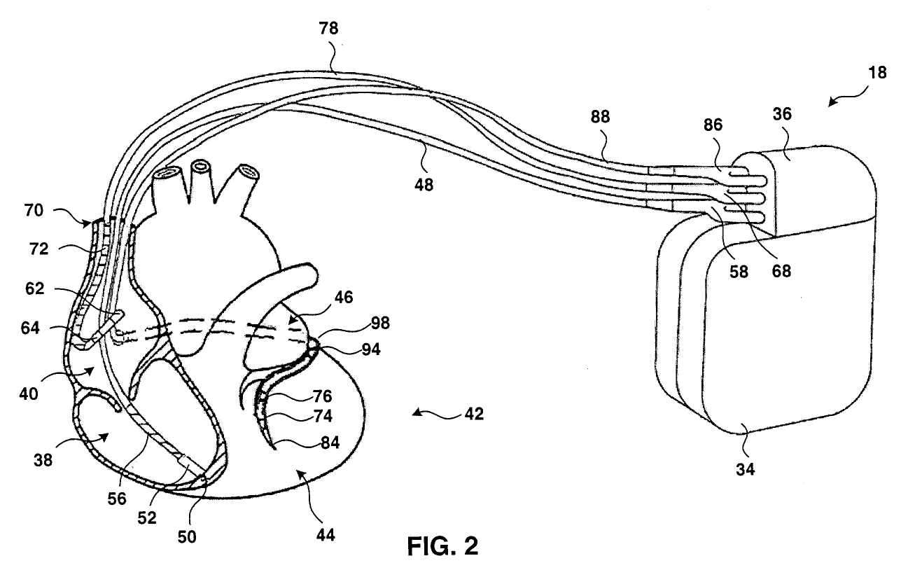

IMD 18 may comprise a multi-chamber pacemaker and may include cardioversion and defibrillation capabilities. Although an exemplary IMD 18 will be described below in connection with FIG. 2, the invention is not limited to the particular IMD shown.

When IMD 18 paces the heart of patient according to the pacing parameter setting, the pacing therapy may affect the hemodynamic operation and/or mechanical function of the heart of patient 20 in a measurable way. For example, is the hemodynamic operation of the heart may be improved, or may be made worse, or may stay substantially the same. Sensor 24 in external device 14 senses the hemodynamic operation, and generates a hemodynamic datum that reflects the hemodynamic operation.

The device 14 communicates the hemodynamic datum to programmer 16, which may set a new pacing parameter as a function of the hemodynamic datum. Programmer 16 programs IMD 18 with the new pacing parameter setting, and IMD 18 implements the programmed pacing parameter setting. External device 14 monitors the hemodynamic operation that results when IMD 18 paces the heart according to the new pacing parameter setting.

In this way, system 10 operates as a closed-loop system, monitoring hemodynamic operation and setting pacing parameters as a function of the hemodynamic operation. Processor 28 in programmer 16 determines which pacing parameters produce desirable hemodynamic results, and may terminate programming when the pacing parameters produce those results.

Although from time to time herein external device 14 is described as external to the body of patient 20 and IMD 18 is implanted in the body of patient 20, the device 14 may be either external or internal (e.g., co-implanted). Moreover, device 14 may share physical components with programmer 16 or IMD 18. In FIG. 1, grouping 12A illustrates an embodiment of the invention in which external device 14 and programmer 16 are both external to the body of patient 20 and share a single housing. In some variations of this embodiment, TX unit 22 may include circuitry to establish a communication link with programmer 16, or TX unit 22 may be omitted as unnecessary.

Grouping 12B illustrates an embodiment in which programmer 16 is implanted in the body of patient 20, and shares the same housing as IMD 18. In some variations of this embodiment, the functionality of TX/RX unit 28 and RX unit 30 may be combined into a single communication unit. Processor 28 in programmer 16 and processor 32 in IMD 18 may also be combined into a single processing unit.

FIG. 2 illustrates one embodiment of IMD 18 that may apply the techniques of the invention. IMD 18 is depicted in conjunction with a human heart 42. IMD 18 is multi-chamber implantable cardioverter-defibrillator (ICD), but the invention is not limited to the particular device depicted in FIG. 2. For example, the IMD 18 may be a single chamber device, may have endocardial, epicardial, transvenous and/or subcutaneous medical electrical leads coupled thereto as well as one or more electrodes surface mounted into a part of a canister or housing for operative circuitry, as is known in the art.

For illustration, a right ventricular lead includes an elongated insulative lead body 48 carrying one or more concentric coiled conductors separated from one another by tubular insulative sheaths. Located adjacent the distal end of lead body 48 are pace/sense electrodes 50, 52. Lead body 48 also includes an elongated coil electrode 56 to apply cardioversion or defibrillation therapy. Each of the electrodes is coupled to one of the coiled conductors within lead body 48. Electrodes 50 and 52 are employed for cardiac pacing and for sensing depolarizations of right ventricle 38. At the proximal end of lead body 48 is a connector 58, which couples the coiled conductors in lead body 48 to a connector module 36.

A right atrial lead includes an elongated insulative lead body 78 carrying one or more concentric coiled conductors separated from one another by tubular insulative sheaths corresponding to the structure of ventricular lead body 48. Located adjacent the J-shaped distal end of lead body 78 are pace/sense electrodes 62, 64, which sense depolarizations of and deliver pacing stimulations to right atrium 40. Elongated coil electrode 72 is provided proximate to the distal end of lead atrial body 78, and is located in right atrium 40 and the superior vena cava 70. At the proximal end of the lead is a connector 68, which couples the coiled conductors in lead body 78 to connector module 36.

A coronary sinus lead shown in FIG. 2 includes an elongated insulative lead body 88 deployed in the great vein 84. Lead body 88 carries one or more coiled conductors coupled to electrodes 74,76,94,98. Electrodes 74,76 are employed for ventricular pacing and for sensing depolarizations of left ventricle 44, and electrodes 94,98 are employed for atrial pacing and for sensing depolarizations of left atrium 46. At the proximal end of the coronary sinus lead is connector 86, which couples the coiled conductors in lead body 88 to connector module 36.

The outward facing portion of housing 34 of IMD 18 may include insulation, such as a coating of parylene or silicone rubber. The outward facing portion of housing 34 may, however, be left uninsulated or some other division between insulated and uninsulated portions may be employed. The uninsulated portion of housing 34 serves as a subcutaneous electrode and a return current path for electrical stimulations applied via other electrodes.

IMD 18 includes an implantable pulse generator (IPG) (not shown in FIG. 2) to generate pacing stimuli, which are delivered to one or more chambers of heart 42. IMD 18 further includes one or more processors (not shown in FIG. 2) that regulate the delivery of pacing pulses. The processors deliver the pacing stimuli according to one or more pacing parameters based on paced/sensed and intrinsic cardiac activity. The pacing parameters govern, for example, the rate or timing of pacing stimuli, and may include one or more pacing intervals as more fully set forth in the Summary portion of this disclosure.

IMD 18 is configured to apply a variety of pacing modes, which includes applying a variety of pacing intervals. IMD 18 may sense or pace one or both atria, and may pace one or more ventricles following an A-V interval. When IMD 18 paces both atria, the atrial paces may be separated by an A-A interval, and when IMD 18 paces both ventricles, the ventricular pacing therapy may be separated by a V-V interval. In accordance with the invention, a programmer may program any of the pacing parameters to a particular setting, and IMD 18 paces the heart according to the pacing parameter settings.

Pacing according to different pacing parameter settings usually affects the hemodynamic operation of heart 42. A sensor 24 in external device 14 monitors the hemodynamic operation, and generates at least one piece of hemodynamic data that directly or indirectly reflects hemodynamic operation and/or mechanical function of a patient 20. External device 14 communicates the hemodynamic datum to programmer 16, which may set a new pacing parameter as a function of the hemodynamic datum and may program IMD 18 with the new pacing parameter setting. As a result of monitoring of the hemodynamic operation of heart 42 by external device 14, IMD 18 may be programmed with one or more pacing parameter settings that result in satisfactory hemodynamic operation. As mentioned, operation of device 14 can be enhanced by receiving information from the IMD 18, and/or the programmer 16, such that the device 14 more readily and efficiently renders measurements related to hemodynamic function.

The invention is not limited to practice with the particular device shown in FIG. 2. For example, a pacemaker that includes a single atrial lead and a single ventricular lead may apply the techniques of the invention to discover an A-V interval that produces good hemodynamic operation. Similarly, a bi-ventricular pacemaker may apply the techniques of the invention to discover a V-V interval that produces good hemodynamic operation. Also, a cardiac stimulation device that provides PESP therapy and/or electrical stimulation therapy to one or more autonomic nerves also benefits from the teaching of the present invention.

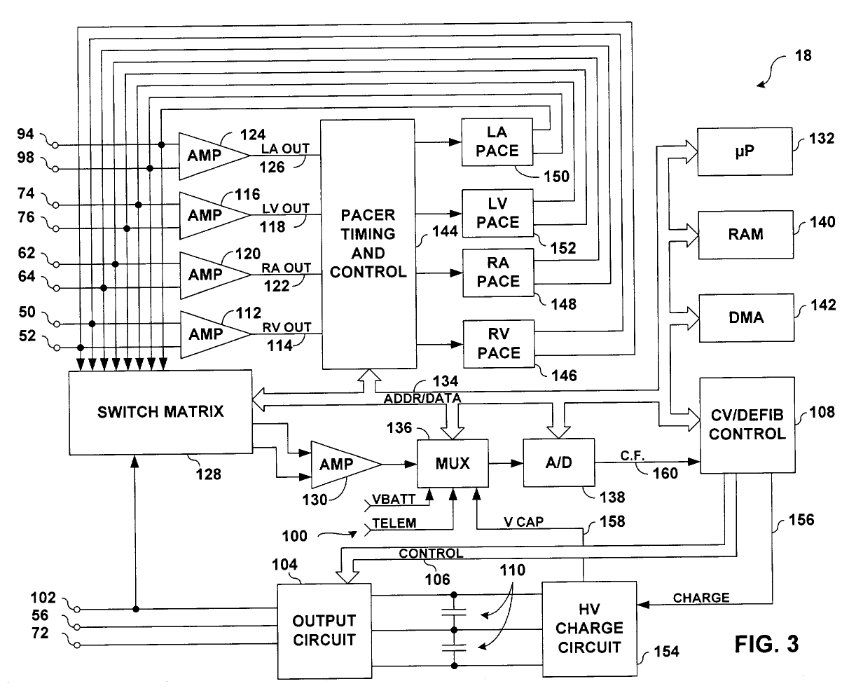

FIG. 3 is a functional schematic diagram of one embodiment of IMD 18. Like FIG. 2, FIG. 3 is exemplary of the type of device that may practice the invention, and the invention is not limited to the particular implementation shown in FIG. 3. On the contrary, the invention may be practiced in a wide variety of device implementations, including devices that lack cardioversion and defibrillation capabilities, and including devices not programmed to address tachyarrhythmias.

As depicted in FIG. 3, IMD 18 includes a telemetry system 100 for wireless communication with a device such as programmer 16. IMD 18 further includes an electrode system described above in connection with FIG. 2. Electrode 102 in FIG. 3 includes the uninsulated portion of the housing 34 of IMD 18. Electrodes 56, 72 and 102 are coupled to high voltage output circuit 104, which includes high voltage switches controlled by cardioversion/defibrillation (CV/defib) control logic 108 via control bus 106. Switches disposed within output circuit 104 determine which electrodes are employed and which electrodes are coupled to the positive and negative terminals of a capacitor bank (which includes capacitors 110) during delivery of defibrillation or cardioversion pulses.

Electrodes 50,52 are located on or in the right ventricle 38 of patient 20 and are coupled to the R-wave amplifier 112, which may take the form of an automatic gain controlled amplifier providing an adjustable sensing threshold as a function of the measured R-wave amplitude. A signal is generated on R-out line 114 whenever the signal sensed between electrodes 50,52 exceeds the sensing threshold.

Similarly, electrodes 94,98 are located proximate to left ventricle 44 and are coupled to the R-wave amplifier 116, which may also take the form of an automatic gain controlled amplifier providing an adjustable sensing threshold as a function of the measured R-wave amplitude. A signal is generated on R-out line 118 whenever the signal sensed between electrodes 94 and 98 exceeds the sensing threshold.

Electrodes 62,64 are located proximate to right atrium 40 and are coupled to the P-wave amplifier 120, which may also take the form of an automatic gain controlled amplifier providing an adjustable sensing threshold as a function of the measured P-wave amplitude. A signal is generated on P-out line 122 whenever the signal sensed between electrodes 62,64 exceeds the sensing threshold.

Similarly, electrodes 74,76 are located proximate to left atrium 46 and are coupled to the P-wave amplifier 124, which may also take the form of an automatic gain controlled amplifier providing an adjustable sensing threshold as a function of the measured P-wave amplitude. A signal is generated on P-out line 126 whenever the signal sensed between electrodes 74,76 exceeds the sensing threshold.

Switch matrix 128 is used to select which of the available electrodes are coupled to amplifier 130 for use in digital signal analysis. Selection of electrodes is controlled by microprocessor 132 via data/address bus 134. Signals from the electrodes selected for coupling to amplifier 130 are provided to multiplexer 136, and thereafter converted to multi-bit digital signals by analog-to-digital (A/D) converter 138, for storage in random access memory (RAM) 140 under control of direct memory access (DMA) circuit 142. Microprocessor 132 may employ digital signal analysis techniques to characterize the digitized signals stored in RAM 140 to recognize and classify the patient's heart rhythm employing any signal processing methodology.

Pacer timing/control circuitry 144 includes programmable digital counters which control the basic time intervals associated with various modes of single- and multi-chamber pacing. Pacer timing/control circuitry 144 also controls escape intervals associated with anti-tachyarrhythmia pacing in both the atrium and the ventricle, employing anti-tachyarrhythmia pacing therapies.

Intervals controlled by pacer timing/control circuitry 144 include, but are not limited to, the A-A interval, A-V interval and V-V interval. The durations of these intervals are typically measured by a filtered signal from one or more sense amplifiers coupled to microprocessor 132, and/or are set in response to programmed or stored data resident in memory 140 and communicated to pacer timing/control circuitry 144 via address/data bus 134. Microprocessor 132 determines durations of the intervals in response to pacing parameter settings received from programmer 16. Pacer timing/control circuitry 144 may determine the amplitude and duration of the cardiac pacing pulses, under control of microprocessor 132. Amplitude and duration of cardiac pacing pulses are examples of additional pacing parameters that may be programmed using the techniques of the invention. In this way, microprocessor 132 and pacer timing/control circuitry 144 cooperate to provide therapeutic electrical stimulation to the heart 42 according to pacing parameter settings received from programmer 16.

As an example, during delivery of pacing therapy escape interval counters within pacer timing/control circuitry 144 are typically reset upon sensing of depolarization wavefronts as indicated by a signals on lines 114,118,122,126 and in accordance with the selected mode of pacing on time-out trigger generation of pacing pulses by pacer output circuitry 146,148,150,152, which are coupled to electrodes 50,52,62,64,74,76,94,98. Escape interval counters are also reset on generation of pacing pulses and thereby control the basic timing of cardiac pacing functions, including anti-tachyarrhythmia pacing. The durations of the intervals defined by escape interval timers are determined by microprocessor 132 via data/address bus 134. The value of the count present in the escape interval counters when reset by sensed R-waves and P-waves may be used to measure the durations of R-R intervals, P-P intervals, P-R intervals and R-P intervals, which measurements are stored in RAM 140 and used to detect the presence of tachyarrhythmias.

Microprocessor 132 may operate as an interrupt driven device, responsive to interrupts from pacer timing/control circuitry 144 corresponding to the occurrence of sensed P-waves and R-waves and corresponding to the generation of cardiac pacing pulses. Those interrupts are provided via data/address bus 134. Any necessary mathematical calculations to be performed by microprocessor 132 and any updating of the values or intervals controlled by pacer timing/control circuitry 144 take place following such interrupts.

In the event that generation of a cardioversion or defibrillation pulse is required, microprocessor 132 may employ an escape interval counter to control timing of such cardioversion and defibrillation pulses, as well as associated refractory and blanking periods, and the like. In response to the detection of atrial or ventricular fibrillation or tachyarrhythmia requiring a cardioversion pulse, microprocessor 132 activates cardioversion/defibrillation control circuitry 108, which initiates charging of high voltage capacitors 110 via charging circuit 154, under the control of high voltage charging control line 156. The voltage on the high voltage capacitors is monitored via VCAP line 158, which is passed through multiplexer 136 and in response to reaching a predetermined value set by microprocessor 132, results in generation of a logic signal on Cap Full (CF) line 160 to terminate charging. Thereafter, timing of the delivery of the defibrillation or cardioversion pulse is controlled by pacer timing/control circuitry 144. Following delivery of the fibrillation or tachycardia therapy microprocessor 132 returns the device to cardiac pacing mode and awaits the next successive interrupt due to pacing or the occurrence of a sensed atrial or ventricular depolarization.

Delivery of cardioversion or defibrillation pulses is accomplished by output circuit 104 under the control of control circuitry 108 via control bus 106. Output circuit 104 determines whether a monophasic or biphasic pulse is delivered, the polarity of the electrodes and which electrodes are involved in delivery of the pulse. Output circuit 104 also includes high voltage switches that control whether electrodes are coupled together during delivery of the pulse.

Although FIGS. 2 and 3 depict two pace/sense electrodes per cardiac chamber, the invention is not limited to two electrodes per chamber. Rather, the invention may be applied to multi-chamber pacing in which there maybe more or fewer than two electrodes per chamber. For example, the invention may be applied to a bi-ventricular pacing system that includes a single electrode in the right ventricle, but three electrodes placed around the left ventricle, such as the left ventricular anterior-septum wall, the left ventricular lateral free wall, and the left ventricular posterior free wall.

Multiple-site electrode placement with respect to a single cardiac chamber may result in more homogenous activation and homogenous mechanical response, which in turn may result in an improved hemodynamic condition for a patient. Consequently, the invention encompasses embodiments in which a single cardiac chamber is responsive to two or more pacing stimuli. In an IMD configured to deliver multiple pacing pulses to a single cardiac chamber, programmer 16 may set the pacing parameter that governs delivery of the pulses to the chamber. Timing intervals between pacing pulses in multiple-electrode systems are further examples of pacing parameters that may be programmed using the techniques of the invention.

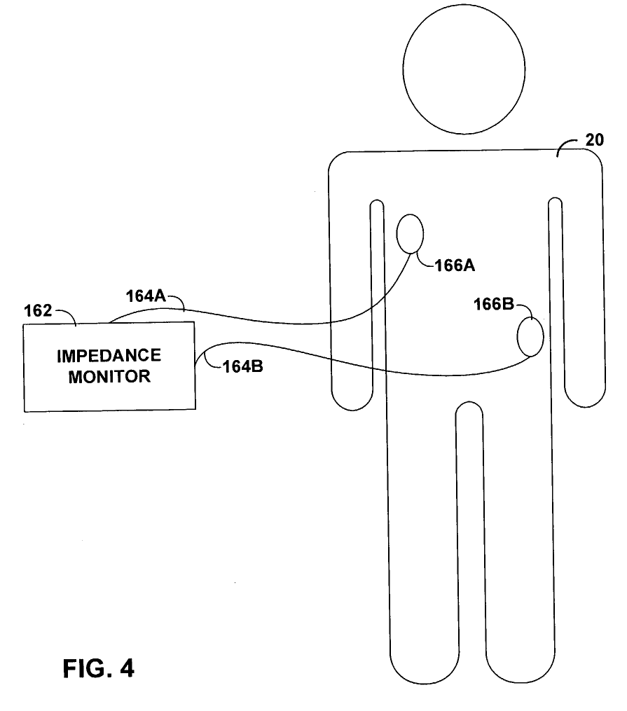

FIG. 4 is a schematic diagram illustrating an exemplary external device to sense the hemodynamic operation of the heart of patient 20 and to generate one or more piece of hemodynamic data as a function of sensed hemodynamic operation. Impedance monitor 162 measures transthoracic impedance, i.e., the impedance across the chest or thorax of patient 20. Impedance monitor 162 is connected to patient 20 via electrodes 166A and 166B and leads 164A and 164B. Impedance monitor 162 measures transthoracic impedance by, e.g., measuring the voltage developed between electrodes 166A and 166B when a known current is applied.

Clinical data have shown that transthoracic impedance varies as a function of cardiac output. In general, an increase in cardiac output results in a decrease in transthoracic impedance, and vice versa. Because transthoracic impedance may vary as a function of other physiological factors such as patient ventilation, impedance monitor 162 ordinarily includes circuitry to filter or otherwise process signals received via electrodes 166A and 166B, to identify physiological factors of interest and ignore other physiological factors.

In response to pacing administered by IMD 18 (not shown in FIG. 4) according to pacing parameter settings, the cardiac output of patient 20 may increase, decrease or stay substantially the same. By measuring changes in transthoracic impedance, impedance monitor 162 monitors changes in cardiac output (or stroke volume). Impedance monitor 162 generates a datum that reflects cardiac output, and supplies that datum to programmer 16. The datum may include a measurement of impedance magnitude, phase angle, resistance, reactance, or any other index that reflects cardiac output. Programmer 16 may, in turn, program a new pacing parameter setting as a function of the datum and may supply the setting to IMD 18. Thereafter, impedance monitor 162 monitors the transthoracic impedance of patient 20 in response to pacing therapy administered by IMD 18 according to the new pacing parameter setting. Also, as previously mentioned, the operation of monitor 162 may be coordinated with the operation or physiologic sensing capabilities of the IMD 18.

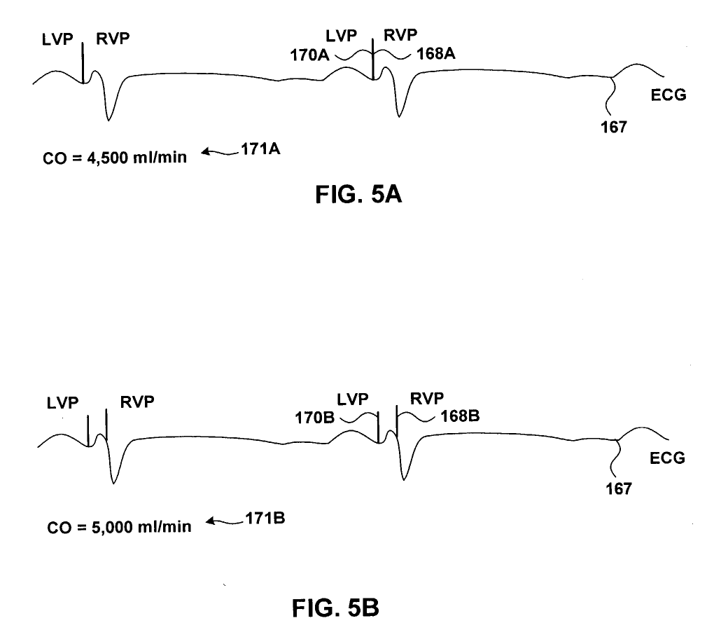

FIGS. 5A and 5B show an electrocardiogram 167 illustrating an exemplary relation between an exemplary hemodynamic datum 171, namely, a cardiac output (CO) or stroke volume measurement, and an exemplary pacing parameter, namely, a V-V interval. FIGS. 5A and 5B depict the timing of a right ventricular pace (RVP) 168 and a left ventricular pace (LVP) 170 with different pacing parameter settings. The use of CO as a hemodynamic datum is for purpose of illustration, and the invention may be practiced with any other indicator of hemodynamic operation and/or mechanical function substantially as set forth in the Summary of this disclosure.

In FIG. 5A, the V-V interval is substantially zero, meaning that IMD 18 delivers RVP 168A and LVP 170A at substantially the same time. Although a coordinated activation of the ventricles generally results in good hemodynamic performance, a simultaneous delivery of right- and left-ventricular paces does not necessarily produce coordinated mechanical function and good hemodynamic performance. In FIG. 5A, the exemplary hemodynamic datum 171A shows that the cardiac output of the patient is about 4.5 liters per minute when the V-V interval is zero.

Upon receiving the hemodynamic datum 171A, programmer 16 resets the V-V interval, and IMD 18 paces the heart according to the new pacing parameter setting. The results of pacing according to the new pacing parameter setting are shown in FIG. 5B. In FIG. 5B, IMD 18 delivers RVP 168B and LVP 170B separated by a time interval, with LVP 170B preceding RVP 168B. In FIG. 5B, the exemplary hemodynamic datum 171B shows that the cardiac output of the patient is about five liters per minute when the heart is paced according to the new pacing parameter setting, which is a substantial improvement in cardiac output.

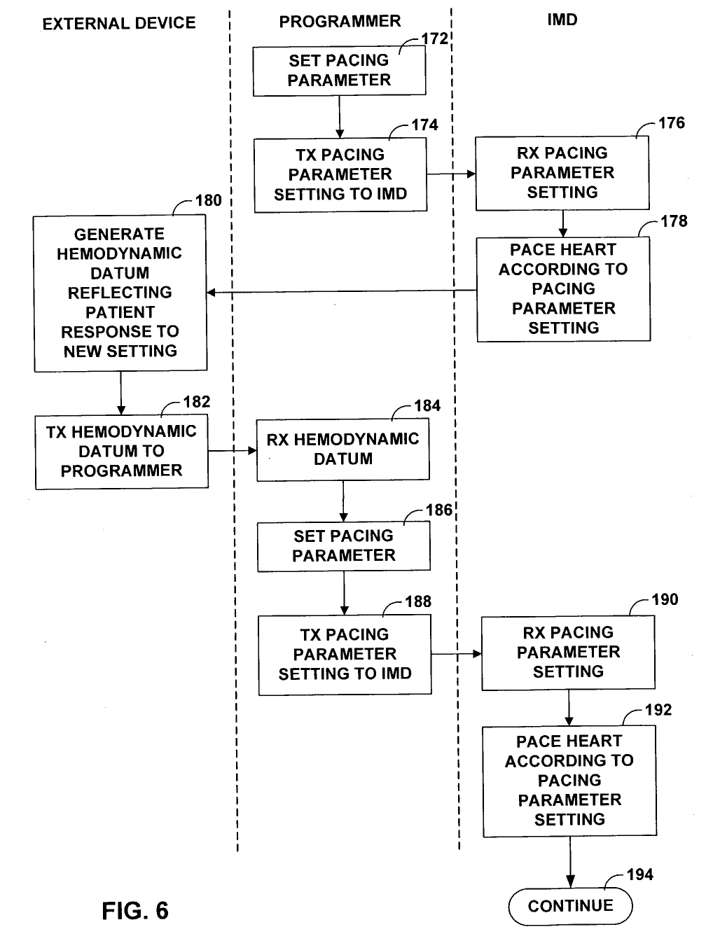

FIG. 6 is a flow diagram illustrating example techniques of the invention. For purposes of FIG. 6, it is assumed that external device 14, programmer 16 and IMD 18 are separate components. At the outset, programmer 16 sets a pacing parameter to an initial setting (172). This initial setting could be a standard default setting, or the initial setting could be a function of patient characteristics, patient history, history of pacing parameters, history of responses to pacing parameters or the like. Programmer 16 transmits the pacing parameter setting to IMD 18 (174), which receives the setting (176).

In response, IMD 18 paces the heart of patient 20 according to the pacing parameter setting (176). In particular, IMD 18 sets or adjusts pacing parameters in accordance with the pacing parameter setting. Pacing in this fashion may continue for several minutes. The cardiovascular system of patient 20, particularly the hemodynamic operation of the heart of patient 20, responds to the pacing over a period of time. As a result, according to the present invention an interval of time (e.g., a number of cardiac cycles or timed period) allows for the patient to respond to the new pacing parameters before attempting to measure the effect on hemodynamic and/or cardiac mechanical performance. Those skilled in the art will appreciate that acute measurements (e.g., stroke volume) may be used to adjust pacing parameters as well as chronic measurements (e.g., cardiac output, or trend information collected over a period of hours, days or weeks).

External device 14 monitors the response, measuring the effect or effects of the pacing parameter setting on patient 20. The device generates at least one hemodynamic datum that reflects the response of patient 20 (180). External device 14 transmits the hemodynamic datum to programmer 16 (182), which receives the hemodynamic datum (184).

In FIG. 6, it is assumed that the hemodynamic datum is unsatisfactory or that further adjustment of one or more pacing parameter may be desired. Accordingly, programmer 16 sets a second pacing parameter as a function of the hemodynamic datum (186), and transmits the second pacing parameter to IMD 18 (188), which receives the second pacing parameter (190). IMD 18 paces the heart of patient 20 according to the second pacing parameter (192).

External device 14, programmer 16 and IMD 18 may repeat this process, measuring the effect or effects of various pacing parameter settings, setting new pacing parameter settings, and pacing the heart according to a new pacing parameter settings. When external device 14 generates a hemodynamic datum indicating that the hemodynamic operation of the patient is satisfactory, programmer 16 discontinues setting new pacing parameter settings. Instead, programmer 16 selects a pacing parameter setting that produced the desired or most beneficial results, and IMD 18 implements the selected pacing parameter setting.

In this way, external device 14, programmer 16 and IMD 18 cooperate to find a set of pacing parameter settings that benefit the patient. Programmer 16 programs IMD 18 with a pacing parameter setting, and receives feedback from hemodynamic measurement device 14 that indicates the effectiveness of the pacing parameter setting. In this way, more effective settings and less effective settings can be identified, and more effective settings can be put into practice.

A number of embodiments of the invention have been described. The invention can be practiced with embodiments other than those disclosed, however. For example, telemetric communication between the device 14 and the IMD 18 may be used to enhance the pacing parameter settings provided by the programmer 16. In addition, although the techniques of the invention are described above as being implemented without human intervention, the invention may also be practiced under the supervision of a clinician. Programmer 16 may receive, for example, input from a clinician in addition to feedback from external device 14 and IMD 18. In addition, the clinician may set the standards for what hemodynamic data indicate more effective hemodynamic operation or less effective hemodynamic operation.

The invention may be embodied as a computer-readable medium that includes instructions for causing a programmable processor, such as processor 28 shown in FIG. 1 pacer timing/control circuitry 144 shown in FIG. 3, to carry out the methods described above. The programmable processor may include one or more individual processors, which may act independently or in concert. A “computer-readable medium” includes but is not limited to read-only memory, Flash memory and a magnetic or optical storage medium. The instructions may be implemented as one or more software modules, which may be executed by themselves or in combination with other software. These and other embodiments are within the scope of the following claims.

1. A method, comprising:
receiving at least one data signal from a hemodynamic monitoring device external to a patient, wherein the at least one data signal relates to hemodynamic function or mechanical cardiac function of the patient; and
programming at least one pacing parameter in an implanted cardiac pacing therapy device as a function of the at least one signal.

2. A method of claim 1, wherein the at least one data signal comprises at least a one of: a stroke volume, a cardiac output, a heart rate.

3. A method of claim 1, wherein programming the at least one pacing parameter comprises setting the at least one pacing parameter to a first setting, and wherein the at least one data signal comprises a first hemodynamic datum, the method further comprising:
receiving a second hemodynamic datum while the at least one pacing parameter is set to the first setting.

4. A method of claim 3, further comprising:
setting the at least one pacing parameter at a second setting as a function of the second hemodynamic datum; and
receiving a third hemodynamic datum while the pacing parameter is at the second setting.

5. A method of claim 1, wherein the at least one pacing parameter comprises a one of: a pacing interval, an intrinsic interval, a combination pacing/intrinsic interval, a blanking period, a refractory period, a cardiac stimulation pulse duration, a cardiac pulse stimulation amplitude, a cardiac stimulation pulse waveform type, a pacing rate, a blanking time period, a refractory time period, an extra-systolic interval, a pacing threshold, a defibrillation threshold, a cardioversion threshold.

6. A method of claim 5, wherein the pacing interval comprises at least one of: an A-V interval, a V-V interval, and an A-A interval.

7. A method comprising:
setting at least one pacing parameter in an implantable medical device to a first setting; and
receiving a hemodynamic datum from a second device external to the body of a patient while the at least one pacing parameter is set to the first setting.

8. A method of claim 7, wherein the hemodynamic datum comprises at least one of a stroke volume datum, a cardiac output datum, and a heart rate datum.

9. A method of claim 7, further comprising setting the at least one pacing parameter to a second setting as a function of the hemodynamic datum.

10. A method of claim 9, wherein the hemodynamic datum is a first hemodynamic datum, the method further comprising:
receiving a second hemodynamic datum from the second device while the pacing parameter is at the second setting.

11. A method of claim 7, wherein the pacing parameter comprises a pacing interval.

12. A method of claim 11, wherein the pacing interval comprises at least one of an A-V interval, a V-V interval, and an A-A interval.

13. A method of claim 7, wherein setting the pacing parameter in the implantable medical device to the first setting comprises transmitting the pacing parameter setting to the implantable medical device.

14. An apparatus comprising a processor that receives a hemodynamic datum from at least one of a first device implanted in the body of a patient and a second device external to the body of the patient, wherein said second device generates a pacing parameter setting for the first device as a function of the hemodynamic datum.

15. An apparatus according to claim 14, further comprising a receiver to receive the hemodynamic datum from the second device.

16. An apparatus according to claim 14, further comprising a transmitter to transmit the pacing parameter setting to the first device.

17. An apparatus according to claim 14, wherein the hemodynamic datum comprises at least one of a stroke volume, a cardiac output, a heart rate.

18. An apparatus according to claim 14, wherein the pacing parameter comprises a pacing interval.

19. An apparatus according to claim 18, wherein the pacing interval comprises at least one of an A-V interval, a V-V interval, and an A-A interval.

20. An apparatus according to claim 14, further comprising a wireless communication circuit among the first device and the second device and wherein said second device further comprises means for sending a data signal from the first device to the second device.

21. An apparatus according to claim 20, wherein the data signal comprises a physiologic data measurement time interval.

22. An apparatus according to claim 20, wherein the first device comprises an implantable cardiac pulse generator and the second device comprises a combination hemodynamic monitor and a programmer for said implantable cardiac pulse generator.

23. An apparatus comprising:
a sensor to measure a hemodynamic datum from a body of a patient;
a transmitter to transmit the hemodynamic datum to a programmer,
wherein the sensor is external to the body of the patient; and
an implantable cardiac pulse generator to provide timed electrical stimulation therapy to the patient based at least in part on the hemodynamic datum.

24. An apparatus according to claim 23, wherein the programmer is internal to the body of the patient.

25. An apparatus according to claim 23, wherein the hemodynamic datum comprises at least one of a stroke volume, a cardiac output, a heart rate.

26. An apparatus according to claim 23, wherein the sensor comprises at least two electrodes to measure at least one transthoracic impedance vector of the patient.

27. A system comprising:
a sensing device external to the body of the patient to measure a hemodynamic datum;
a programmer to generate a pacing parameter setting as a function of the hemodynamic datum; and
an implantable medical device to apply pacing stimuli to a heart according to the pacing parameter setting.

28. A system according to claim 27, wherein the programmer is external to the body of the patient.

29. A system according to claim 27, wherein the hemodynamic datum comprises at least one of a stroke volume datum, a cardiac output datum, a heart rate datum.

30. A system according to claim 27, wherein the pacing parameter comprises a pacing interval.

31. A system according to claim 30, wherein the pacing interval comprises at least one of an A-V interval, a V-V interval, an A-A interval.

32. A system according to claim 27, the sensing device comprising at least one electrode to measure an impedance of the patient.

33. A computer-readable medium comprising instructions for causing a programmable processor to:
receive a hemodynamic datum from a first device external to the body of a patient; and
set a pacing parameter in a second device implanted in the patient as a function of the hemodynamic datum.

34. A medium according to claim 33, wherein the hemodynamic datum comprises at least one of stroke volume, cardiac output, and heart rate.

35. A medium according to claim 33,
wherein the instructions causing the processor to set the pacing parameter in the second device comprise instructions causing the processor to set the pacing parameter at a first setting, and wherein the hemodynamic datum is a first hemodynamic datum, the instructions further causing the processor to receive a second hemodynamic datum while the pacing parameter is at the first setting.

36. A medium according to claim 35, the instructions further causing the processor to:
set the pacing parameter in the second device at a second setting as a function of the second hemodynamic datum; and
receive a third hemodynamic datum while the pacing parameter is at the second setting.

37. A medium according to claim 33, wherein the pacing parameter comprises a pacing interval.

38. A medium according to claim 37, wherein the pacing interval comprises at least one of an A-V interval, a V-V interval, and an A-A interval.

39. A computer-readable medium comprising instructions for causing a programmable processor to:
set a pacing parameter in an implantable medical device to a first setting; and
receive a hemodynamic datum from a second device external to the body of a patient while the pacing parameter is at the first setting.

40. A medium according to claim 39, wherein the hemodynamic datum comprises at least one of a stroke volume, a cardiac output, a heart rate.

41. A medium according to claim 39, the instructions further causing the processor to set the pacing parameter to a second setting as a function of the hemodynamic datum.

42. A medium according to claim 41, wherein the hemodynamic datum is a first hemodynamic datum, the instructions further causing the processor to receive a second hemodynamic datum from the second device while the pacing parameter is at the second setting.

43. A medium according to claim 39, wherein the pacing parameter comprises a pacing interval.

44. A medium according to claim 43, wherein the pacing interval comprises at least one of an A-V interval, a V-V interval, and an A-A interval.

45. A medium according to claim 39, wherein the instructions causing the processor to set the pacing parameter in the implantable medical device to the first setting comprise instructions causing the processor to transmit the pacing parameter setting to the implantable medical device.

46. A system comprising:
means for measuring at least one hemodynamic datum of a patient with a first external device;
means for programming at least one pacing parameter as a function of the at least one hemodynamic datum with a second external device; and
means for applying cardiac pacing therapy according to the at least one pacing parameter.

47. A system according to claim 46, wherein the means for applying cardiac pacing therapy comprises an implantable medical device.

48. A system according to claim 47, wherein the means for programming comprises wirelessly acquiring data from the means for measuring.

49. A system according to claim 48, wherein the means for measuring further comprises means for receiving at least one timing signal from the implantable medical device so that the means for measuring extracts a measurement during a predetermined period of time.

50. A system according to claim 46, wherein said at least one hemodynamic datum comprises at least a one of:
a stroke volume datum, a cardiac output datum, a heart rate datum, an ECG/EGM recording datum, a heart sound datum, a blood pressure datum, a blood flow datum, a temperature datum, a tissue impedance datum, a trans-thoracic impedance datum, a body fluid datum, a tissue fluid saturation datum, a circulation delay time datum, a cardiac tissue contractility index datum, a cardiac mechanical restitution datum, a recirculation fraction datum, an ejection fraction datum, an acceleration datum for a moving part of a heart, a volumetric datum.

51. A system according to claim 46, wherein said at least one pacing parameter comprises at least a one of:
a timed, device-related cardiac pacing interval;
a sensed, intrinsic cardiac interval;
a combination pacing/intrinsic interval;
a blanking period;
a refractory period;
a pacing threshold;
a defibrillation threshold;
a cardioversion threshold.
